(19) United States
(12) Patent Application Publication
(10) Pub. No.: US 2003/0032847 A1
(43) Pub. Date: Feb. 13, 2003

(54) Process for hydrogenating/dehalogenating polyalphaolefin polymer, the resulting polymer and lubricant containing same

(75) Inventor: Michael P. Reynolds, Naugatuck, CT (US)

Correspondence Address: Daniel Reitenbach, CROMPTON CORPORATION, Benson Road, Middlebury, CT, 06749, US

(73) Assignee: CROMPTON CORPORATION

(21) Appl. No.: 09/873,848

(22) Filed: Jun. 4, 2001

(51) Int. Cl. C07C 5/05; C07C 5/00

(52) U.S. Cl. 585/271; 585/273; 585/274; 208/14; 208/262.1

(57) ABSTRACT

A substantially hydrogenated and/or substantially dehalogenated polyalphaolefin homo- or copolymer is obtained by a hydrogenation/dehalogenation process employing hydrogen and a substantially amorphous catalyst containing a metal component on an inorganic material based support. The substantially hydrogenated/dehalogenated polyalphaolefin homo- or copolymers provided herein are useful for manufacturing a variety of products including lubricating oils in which the polyalphaolefin functions as a viscosity modifier.

### BACKGROUND OF THE INVENTION

1. Field of the Invention

This invention relates generally to a process for substantially hydrogenating and/or dehalogenating a polyalphaolefin homopolymer, e.g., 1-decene, or copolymer, e.g., one derived from 1-decene, employing hydrogen and an amorphous hydrogenation/dehalogenation catalyst therefor, to the resulting polymer and to a lubricant composition in which the substantially hydrogenated/dehalogenated polyalphaolefin functions as a viscosity modifier.

2. Description of the Related Art

Hydrogenation is a well-established process for use in both the chemical and petroleum refining industries. In general, hydrogenation has been carried out in the presence of a hydrogenation catalyst containing a Group VIII metal, e.g., nickel, platinum, palladium, rhodium, iridium, etc., on a crystalline based porous support. See, e.g., U.S. Pat. No. 5,573,657.

Hydrogenation is frequently used in petroleum refining to improve the qualities of lubricating oils, both of natural and synthetic origin. Generally, hydrogenation is employed to reduce residual unsaturation in the lubricating oil, and to remove heteroatom-containing impurities and color bodies. The removal of impurities and color bodies is of particular significance for mineral oils which have been subjected to hydrocracking or catalytic dewaxing. For both hydroprocessed mineral and synthetic stocks, the saturation of lube boiling range olefins is a major objective. One class of synthetic hydrocarbon lubricants which have achieved importance in the lubricating oil market are polyolefins. These materials are typically produced by the polymerization of alpha-olefins ranging from 1-octene to 1-dodecene, although polymers of lower olefins such as ethylene and propylene may also be used including ethylene with higher olefins.

### SUMMARY OF THE INVENTION

It is an object of the present invention to provide a hydrogenation and/or dehalogenation process for producing a substantially hydrogenated and/or dehalogenated polyalphaolefin polymer employing hydrogen and a substantially amorphous hydrogenation/dehalogenation supported catalyst containing at least a metal component on an inorganic material based support.

It is a further object of the invention to provide such a process for the hydrogenation and/or dehalogenation of alphaolefins to provide substantially saturated and/or dehalogenated polyalphaolefin homopolymers, e.g., 1-decene, or copolymers, e.g., one derived from 1-decene.

Additional objects of the invention include providing a polyalphaolefin homo- or copolymer possessing a combination of low iodine number (I₂) and low halogen content, the process comprising contacting at least one polyalphaolefin, e.g., one having from 2 to about 20 carbon atoms, under hydrogenating and/or dehalogenating conditions with hydrogen and a substantially amorphous hydrogenation/dehalogenation supported catalyst comprising a metal component on an inorganic material based support.

### DESCRIPTION OF THE PREFERRED EMBODIMENT

The resulting polyalphaolefin polymers obtained from the process of this invention are substantially saturated, i.e., one possessing a low iodine number, and/or substantially dehalogenated, i.e., one possessing a low halogen content, e.g. bromine, chlorine, fluorine etc., and can be obtained by contacting at least one polyalphaolefin under hydrogenating and/or dehalogenating conditions in the presence of hydrogen and a substantially amorphous hydrogenation/dehalogenation supported catalyst containing at least a metal component on an amorphous inorganic material based support.

The α-olefins suitable for use in the preparation of the substantially hydrogenated and/or dehalogenated polyalphaolefin polymers obtained herein contain from 2 to about 20 carbon atoms and preferably from about 6 to about 12 carbon atoms which are utilized after polymerization in the process of the present invention. Suitable α-olefins include ethylene, propylene, 2-methylpropene, 1-butene, 3-methyl-1-butene, 1-pentene, 4-methyl-1-pentene, 1-hexene, 1-heptene, 1-octene, 1-nonene, 1-decene, 1-undecene, 1-dodecene, 1-tridecene, 1-tetradecene, 1-pentadecene, 1-hexadecene, 1-heptadecene, 1-octadecene, 1-nonadecene, 1-eicosene and the like and vinyl aromatic monomers such as styrene, α-methyl styrene and the like and mixtures thereof. Preferred α-olefins for use herein are 1-octene, 1-decene and 1-dodecene with 1-decene being most preferred.

The polyalphaolefins for use herein are advantageously obtained employing any conventional polymerization process known to one skilled in the art, e.g., by polymerization either thermally or catalytically in the presence of, for example, a di-tertiary alkyl peroxide or a Friedel-Crafts catalyst. The preferred polyalphaolefin homopolymer for use herein will contain up to about 100 weight percent 1-decene while the preferred polymerized polyalphaolefin copolymer can contain up to about 95, preferably from about 20 to about 90, and more preferably from about 30 to about 85, weight percent 1-decene, the balance being other α-olefin(s).

The amorphous hydrogenation/dehalogenation supported catalyst for use herein is formed from at least metal component on an amorphous inorganic material based support. Suitable metals useful in forming the supported catalyst are metals of Group VIII of the Period Table of the Elements such as iron (Fe), cobalt (Co), nickel (Ni), ruthenium (Ru), rhodium (Rh), palladium (Pd), rhenium (Re), osmium (Os), iridium (Ir), platinum (Pt), and the like and salts thereof and combinations thereof. A preferred metal component for use herein is palladium.

Suitable inorganic support materials for forming the supported catalyst include amorphous metal oxides such as, for example, alumina, silica-alumina, titanium and the like with silica-alumina being preferred. The presently preferred catalyst for use herein is palladium supported on silica-alumina for it is readily available, e.g., those commercially available from Süd-Chemie AG and Johnson Matthey. Generally, the supported catalyst of this invention should have particle size distribution with some particles greater than about 250 microns (μ) and some particles less than about 75μ. The preferred catalyst should have about 2% of the particles greater than about 250μ, with about 10% greater than about 170μ, from about 55 to about 90% of the catalyst should have particles from about 106 to about 250μ from about 5 to about 30% should be from about 75 to about 130μ, and no more than about 10% should be less than about 75μ. Although the particle distribution has been described in its preferred form with a certain degree of particularity, obviously minor changes and variations are possible therein and will be apparent to those skilled in the art.

Generally, the supported catalyst can be formed employing conventional techniques known in the art. For example, the supported catalyst can be prepared by dry mixing the components or by immersing or impregnating the support which comprises filling the pore volume of the support with a solution or dispersion of the metal component in elemental form or in the form of reducible compounds thereof to physically carry the metal component. Alternatively, a spray method can be utilized which comprises spraying the metal component to the support. The supported catalysts can be penetrated with hydrogen to reduce the metal component, or such reduction can be achieved in the hydrogenation reactor. After the metal compound has been deposited on the support, the supported catalyst can thereafter be dried and calcined. Each drying step can be performed at temperatures of, for example, within the range of from about 100° to about 300° C. for a time sufficient to remove substantially all water (i.e., in the case of use of aqueous solutions of the foregoing Group VIII metals) or at a temperature above the boiling point of a solvent, when employed, to about 300° C., for removal of any other selected solvent used during the impregnation or deposition of the metals, optionally together with passing of an inert gas such as, e.g., nitrogen, over the metal's surface to facilitate the removal of the water or solvent. The calcining temperatures and times used can be those as described above.

The catalyst will advantageously contain the metal component in range of from about 0.01 to about 5 weight percent, preferably from about 0.05 to about 3 weight percent and most preferably from about 1.5 to about 2.5 weight percent, based on the total catalyst weight basis.

The hydrogenation and/or dehalogenation process of the aforementioned polyalphaolefins in the presence of hydrogen and the catalyst herein can be carried out in any known manner, e.g., in the liquid phase, i.e., in a solution or slurry process, or in a gas or suspension process, either continuously, semi-batch or in batch. Generally, these processes are carried out at temperatures in the range of from about 50° C. to about 350° C., and pressures from about 50 psig to about 500 psig. The time period for hydrogenation and/or dehalogenation will depend upon the temperatures and pressures employed and can take from about 0.5 to about 12 hours.

Due to the nature of the final polyalphaolefin, hydrogenation and/or dehalogenation can be carried out in liquid polyalphaolefin and in the absence of solvent or, if desired, in the presence of solvent. Dilution solvents that can be employed include straight and branched chain hydrocarbons such as the butanes, the pentanes, the hexanes, the heptanes, the octanes, and the like, cyclic and alicyclic hydrocarbons such as cyclopentane, cyclohexane, cycloheptane, methyl-cyclopentane, methylcyclohexane, methylcycloheptane and the like, and alkyl-substituted aromatic compounds such as toluene, xylene, and the like and mixtures thereof.

A typical batch hydrogenation and/or dehalogenation process can be carried out by first introducing the polyalphaolefin, e.g., 1-decene, into a suitable vessel such as, for example, a stirred tank reactor. The reactor is then charged with a measured amount of catalyst and hydrogen and brought up to the desired temperature, e.g., from about 50 to about 350° C., and preferably from about 150 to about 250° C. By carrying out the hydrogenation and/or dehalogenation reaction in the presence of hydrogen and employing the catalyst herein, the resulting polyalphaolefins of this invention are substantially saturated, i.e., one possessing a low iodine value, e.g., an iodine number of from about 0.5 to about 10, preferably from about 1 to about 8, and most preferably from about 2 to about 5, and can also be substantially dehalogenated, i.e., one possessing a low halogen content (e.g. bromine, chlorine, or fluorine) value, e.g., a halogen content of from about 1 to about 200 ppm, preferably from about 3 to about 100 ppm and most preferably from about 5 to about 50 ppm.

The catalyst is typically added in the required amounts, e.g., from about 0.01 wt % to about 1 wt % and preferably from about 0.05 wt % to about 0.7 wt %, based on the total weight of the liquid phase, to the liquid phase in the reactor to form a slurry. The rate of hydrogenation and/or dehalogenation is controlled by the concentration of the catalyst, hydrogen pressure, and polyalphaolefin. The reactor temperature is controlled by means of cooling coils, etc., and the total pressure in the reactor is maintained by a constant flow of hydrogen, inert gas, e.g., nitrogen, or a combination thereof. After hydrogenation and/or dehalogenation is complete, the reactor is depressurized. The catalyst and polyalphaolefin can be separated from the slurry employing conventional techniques, e.g., filtration or settling. Once the catalyst is separated by conventional techniques it can be recovered, recycled and/or reused. The resulting polyalphaolefin can then be further processed as desired.

The resulting polyalphaolefins possessing the advantageous properties can be exploited in a variety of products such as, for example, products which require a viscous oil or an inert material with fluid properties such as dispersants, heat transfer fluids, cosmetics or other such consumer products, and the like. Additionally, the products of this invention can be used in grafting applications to produce functionalized low molecular weight polymers. The polyalphaolefin polymers of this invention are particularly useful as a viscosity modifier for lubricating oils wherein the polymer is employed in a viscosity-modifying amount. Concentrations of from about 1 to about 99 weight percent based on the total weight of the lubricating oil composition can be used. Preferably, the concentration is from about 5 to about 85 weight percent.

In general, mineral oils, both paraffinic, naphthenic and mixtures thereof, including those oils defined as American Petroleum Institute Groups I, II, and III can be employed as the lubricant vehicle, and can be any suitable lubricating viscosity range, as for example, from about 2 cSt at 100° C. to about 1,000 cSt at 100° C. and preferably from about 2 to about 100 cSt at 100° C. These oils can have viscosity indexes preferably ranging to about 180. The average molecular weights of these oils can range from about 250 to about 800. Where synthetic oils are employed, they can include, but are not limited to, polyisobutylene, polybutenes, hydrogenated polydecenes, polypropylene glycol, polyethylene glycol, trimethylpropane esters, neopentyl and pentaerythritol esters, di(2-ethylhexyl) sebacate, di(2-ethylhexyl) adipate, dibutyl phthalate, fluorocarbons, silicate esters, silanes, esters of phosphorus-containing acids, liquid ureas, ferrocene derivatives, hydrogenated synthetic oils, chain-type polyphenyls, siloxanes and silicones (polysiloxanes), alkylsubstituted diphenyl ethers typified by a butyl-substituted bis(p-phenoxy phenyl) ether, and phenoxy phenylethers.

The lubricating oil compositions herein can also contain one or more other materials. For example, detergents, corrosion inhibitors, oxidative inhibitors, dispersants, pour point dispersants, anti-foaming agents, anti-wear agents, other viscosity modifiers, friction modifiers and the like at the usual levels in accordance with well known practice. Other materials which can be employed herein include extreme pressure agents, low temperature properties modifiers and the like can be used as exemplified respectively by metallic penates or sulfonates, polymeric succinimides, non-metallic or metallic phosphorodithioates and the like, at the usual levels in accordance with well known practice. These materials do not detract from the value of the compositions of this invention, rather the materials serve to impart their customary properties to the particular compositions in which they are incorporated.

The following non-limiting examples are illustrative of the method of the present invention.

To determine the properties of the polyolefins obtained in the examples below, the following procedures were used.

### Unsaturation Determination by Iodine Number

The amount of unsaturation in the polyolefins was determined by measurement of the Iodine Number (I₂ No.) which is defined as the number of grams of iodine that add to 100 grams of sample. Only halogen that combines with a sample by way of addition to double bonds is a true measurement of unsaturation. Substitution reactions and, to a lesser extent, splitting-out reactions contribute to some error in the determination. In this method, the slow rate of addition of iodine to double bonds is catalyzed by Mercuric Acetate allowing the reaction to be completed in about one hour where the effects of the slower substitution and splitting-out reactions are minimized. The method was adapted from Gallo et al., “Unsaturation in Isoprene-Isobutylene Copolymers”, Industrial and Engineering Chemistry, Vol. 40, (1948) pp. 1277-1280. An Iodine Number of less than, for example, about 10, is considered substantially saturated.

### Dehalogenation Determination by Halogen Content

The halogen content of the polyolefins was determined by two methods.

Samples of PAO were decomposed by combustion in an oxygen bomb made by The Parr Instrument Corporation. The combustion products are absorbed by aqueous solutions. The halogen content is then determined by using a previously calibrated Specific Ion Electrode.

In the second method, the analysis was done using an Oxford Instrument Company Model Lab-X3000 X-ray Fluorescence Spectrometer. Calibration curves were prepared by making solutions of a non-volatile organo halide compound in mineral oil.

Any other acceptable method of halogen determination should be suitable.

### EXAMPLE 1

A 1-gallon Hastelloy C autoclave was loaded with 1400 g (˜1750 mls) of 1-decene homopolymer having an iodine number of 28 and a bromine content of 480 ppm. The vessel was then charged with 2.472 g dry Süd-Chemie MGS5 hydrogenation/dehalogenation catalyst of 2% Pd on sodium silicoaluminate available from Süd-Chemie AG and sealed. Next, the vessel was purged twice with nitrogen and vented, once with hydrogen and vented, and then pressured to 200 psig of hydrogen. After a successful pressure test the vessel was agitated and heated under hydrogen to 235° C. for 5.0 hours. The vessel was equipped with a dip tube containing a 0.5 micron sintered metal filter. Sampling was completed hourly using the dip tube.

The final product possessed an iodine number of 3.1 and a bromine content of 10 ppm.

### EXAMPLE 2

A 1-gallon Hastelloy C autoclave was loaded with 1400 g (˜1750 mls) of 1-decene homopolymer having an iodine number of 37 and a bromine content of 671 ppm. The vessel was then charged with 2.472 g dry Süd-Chemie MGS5 hydrogenation/dehalogenation catalyst of 2% Pd on sodium silicoaluminate available from Süd-Chemie AG and sealed. Next, the vessel was purged twice with nitrogen and vented, once with hydrogen and vented, and then pressured to 200 psig of hydrogen. After a successful pressure test the vessel was agitated and heated under hydrogen to 235° C. for 5.0 hours. The vessel was equipped with a dip tube containing a 0.5 micron sintered metal filter. Sampling was completed hourly using the dip tube.

The final product possessed an iodine number of 2.0 and a bromine content of 12 ppm.

### EXAMPLE 3

Employing essentially the same procedure as in Example 1, and the same materials as in Example 2, except this example used 2.472 g dry hydrogenation/dehalogenation catalyst of 2% Pd on silicoaluminate catalyst available from Johnson Matthey. The final product possessed an iodine value of 0.6 and a bromine content of <2 ppm.

### EXAMPLE 4

Employing essentially the same procedure as in Example 1, and the same materials as in Example 2, except this example used 2.472 g dry hydrogenation/dehalogenation catalyst of 2% Pd on alumina catalyst available from Johnson Matthey. The final product possessed an iodine number of 1.2 and a bromine content of 14 ppm.

### EXAMPLE 5

A 1-gallon Hastelloy C autoclave was loaded with 1400 g (˜1750 mls) of 1-decene homopolymer having an iodine number of 28 and a bromine content of 480 ppm. The vessel was then charged with 9.887 g dry Süd-Chemie MGS5 hydrogenation/dehalogenation catalyst of 2% Pd on sodium silicoaluminate available from Süd-Chemie AG and sealed. This catalyst level was four times the level of the catalyst of Example 1. Next, the vessel was purged twice with nitrogen and vented, once with hydrogen and vented, and then pressured to 200 psig of hydrogen. After a successful pressure test the vessel was agitated and heated under hydrogen to 235° C. for 5.0 hours. The vessel was equipped with a dip tube containing a 0.5 micron sintered metal filter. Sampling was completed hourly using the dip tube. After the last sample was taken, the vessel was cooled, vented of hydrogen, and purged with nitrogen. The remaining polyalphaolefin in the autoclave was removed by applying nitrogen pressure to push the material out of the dip tube and collected in a flask. A small heal remained in the autoclave with original catalyst charged. An additional 1400 g of unhydrogenated 1-decene homopolymer was charged back through the dip to ensure removal of the catalyst from the sintered metal filter. The vessel was purged twice with nitrogen and vented, once with hydrogen and vented, and then pressured to 200 psig of hydrogen. After a successful pressure test the vessel was agitated and heated under hydrogen to 235° C. for 5.0 hours. This process was repeated a total of four times. The catalyst originally charged at the beginning was used for 5 hydrogenations and dehalogenations. The results of these tests are set forth below in Table I.

TABLE I

| Hydrogenation/ | | Final Bromine |
|---|---|---|
| Dehalogenation | Final Iodine No. | Content (ppm)# |
| 1^{a} | 1.4 | 13 |
| 2^{b} | 1.6 | <2 |
| 3^{c} | 0.6 | 4 |
| 4^{d} | 0.5 | 25 |
| 5^{d} | 0.8 | 21 |

^{a}This is the iodine number and bromine content after 2.0 hours.
^{b}This is the iodine number and bromine content after 3.0 hours.
^{c}This is the iodine number and bromine content after 4.0 hours.
^{d}This is the iodine number and bromine content after 5.0 hours

### EXAMPLE 6

Example 4 was repeated with the results being set forth below in Table II.

TABLE II

| Hydrogenation/ | | Final Bromine |
|---|---|---|
| Dehalogenation | Final Iodine # | Content (ppm)# |
| 1^{a} | 1.7 | 5 |
| 2^{b} | 1.2 | 14 |
| 3^{c} | 0.6 | 21 |
| 4^{d} | 0.6 | 21 |
| 5^{d} | 1.1 | 31 |

^{a}This is the iodine number and bromine content after 2.0 hours.
^{b}This is the iodine number and bromine content after 3.0 hours.
^{c}This is the iodine number and bromine content after 4.0 hours.
^{d}This is the iodine number and bromine content after 5.0 hours

### EXAMPLE 7

A 1-gallon Hastelloy C autoclave was loaded with 1400 g (˜1750 mls) of 1-decene homopolymer having an iodine number of 28 and a bromine content of 480 ppm. The vessel was initially charged with 4.935 g dry Süd-Chemie MGS5 hydrogenation/dehalogenation catalyst of 2% Pd on sodium silicoaluminate available from Süd-Chemie AG and sealed. This catalyst level was twice the level of the catalyst of Example 1. The vessel was purged twice with nitrogen and vented, once with hydrogen and vented, and pressured to 200 psig of hydrogen. After a successful pressure test the vessel was agitated and heated under hydrogen to 235° C. for 3.0 hours. The vessel was equipped with a dip tube containing a 0.5 micron sintered metal filter. Sampling was completed hourly using the dip tube. After the last sample was taken, the vessel was cooled, vented of hydrogen, and purged with nitrogen. The remaining polyalphaolefin in the autoclave was removed by applying nitrogen pressure to push the material out of the dip tube and collected in a flask. A small heal remained in the autoclave with original catalyst charged. An additional 1400 g of unhydrogenated 1-decene homopolymer was charged back through the dip to ensure removal of the catalyst from the sintered metal filter. In each subsequent hydrogenation the autoclave was charged with an additional amount of catalyst (0.5 times that of Example 1) of 1.236 g dry of the Süd-Chemie MSG5. The vessel was purged twice with nitrogen and vented, once with hydrogen and vented, and then pressured to 200 psig of hydrogen. After a successful pressure test the vessel was agitated and heated under hydrogen to 235° C. for 3.0 hours. This process was repeated a total of four times. The catalyst originally charged at the beginning was used for 5 hydrogenations and dehalogenations. The results of these tests are set forth below in Table III.

TABLE III

| Hydrogenation/ | Catalyst Charged dry (g) | | | Final | Final Bromine |
|---|---|---|---|---|---|
| Dehalogenation | Fresh | Recycled | Total | Iodine No.^{a} | Content (ppm)^{b} |
| 1 | 4.935 | 0 | 4.935 | 1.3 | 6 |
| 2 | 1.236 | 4.935 | 6.171 | 2.0 | 9 |
| 3 | 1.236 | 6.171 | 7.407 | 2.2 | 6 |
| 4 | 1.236 | 7.407 | 8.643 | 2.6 | 12 |
| 5 | 1.236 | 8.643 | 9.879 | 2.4 | 7 |

^{a}This is the iodine number after 5.0 hours.
^{b}This is the bromine content after 5.0 hours.

### EXAMPLE 8

Employing essentially the same procedures as in Example 1, and the same materials as in Example 2, except this example used a hydrogenation/dehalogenation catalyst of 5% Pd on sodium silicoaluminate catalyst available form Süd-Chemie AG. The final product possessed an iodine number of 8.0 and a bromine content of 52 ppm.

Although the invention has been described in its preferred form with a certain degree of particularity, obviously many changes and variations are possible therein and will be apparent to those skilled in the art after reading the foregoing description. It is therefore to be understood that the present invention may be presented otherwise than as specifically described herein without departing from the spirit and scope thereof.

What is claimed is:

1. A process for the hydrogenation and/or dehalogenation of α-olefin to provide a substantially hydrogenated and/or substantially dehalogenated polyalphaolefin homo- or copolymer, the process comprising hydrogenating and/or dehalogenating at least one polymerized α-olefin under catalytic hydrogenation and/or dehalogenation conditions in the presence of hydrogen and a catalytically effective amount of a substantially amorphous hydrogenation/dehalogenation catalyst comprising a metal component on an inorganic material based support.

2. The process of claim 1 wherein the α-olefin contains from 2 to about 20 carbon atoms.

3. The process of claim 1 wherein the α-olefin contains from about 6 to about 12 carbon atoms.

4. The process of claim 1 wherein the α-olefin is 1-decene.

5. The process of claim 1 wherein the metal component of the catalyst is one or more Group VIII metals of the Periodic Table selected from the group consisting of Fe, Co, Ni, Ru, Rh, Pd, Os, Ir, Pt, and salts thereof.

6. The process of claim 1 wherein the inorganic support is a material selected from the group consisting of silica, alumina and silica-alumina.

7. The process of claim 1 wherein the amorphous hydrogenation/dehalogenation catalyst is palladium on a silica-alumina support.

8. A substantially hydrogenated and/or substantially dehalogenated polyalphaolefin homo- or copolymer obtained from the polymerization of at least one α-olefin, the process comprising hydrogenating and/or dehalogenating the α-olefin under catalytic hydrogenating and/or dehalogenating conditions in the presence of hydrogen and a catalytically effective amount of a substantially amorphous hydrogenating/dehalogenating catalyst comprising a metal component on an inorganic material based support.

9. The polyalphaolefin of claim 8 wherein the α-olefin contains from 2 to about 20 carbon atoms.

10. The polyalphaolefin of claim 8 wherein the α-olefin is selected from the group consisting of 1-octene, 1-decene, 1-dodecene and combinations thereof.

11. The polyalphaolefin of claim 8 wherein the metal component of the catalyst is one or more Group VIII metals of the Periodic Table selected from the group consisting of Fe, Co, Ni, Ru, Rh, Pd, Os, Ir, Pt, and salts thereof.

12. The polyalphaolefin of claim 8 wherein the inorganic support is a material selected from the group consisting of silica, alumina and silica-alumina.

13. The polyalphaolefin of claim 8 wherein the amorphous hydrogenation/dehalogenation catalyst is palladium on a silica-alumina support.

14. The polyalphaolefin of claim 8 possessing an iodine number of from about 0.5 to about 10.

15. The polyalphaolefin of claim 8 possessing a halogen content of from about 1 to about 200 ppm.

16. The polyalphaolefin of claim 8 possessing an iodine number of from about 0.5 to about 10 and a halogen content of from about 1 to about 200 ppm.

17. The polyalphaolefin of claim 13 possessing an iodine number of from about 1 to about 8 and a halogen content of from about 3 to about 100 ppm.

18. A lubricating oil composition comprising a lubricating oil and a viscosity-modifying amount of the polyalphaolefin of claim 8.

19. A lubricating oil composition comprising a lubricating oil and a viscosity-modifying amount of the polyalphaolefin of claim 16.

20. A lubricating oil composition comprising a lubricating oil and a viscosity-modifying amount of the polyalphaolefin of claim 17.
